(19) United States
(12) Patent Application Publication
(10) Pub. No.: US 2010/0204624 A1
(43) Pub. Date: Aug. 12, 2010

(54) ENDO-BUCCAL DEVICE FOR TACTILE STIMULATION AND ACTUATION

(75) Inventors: Nicolas Vuillerme, Francin (FR); Yohan Payan, Allevard (FR); Jacques Demongeot, Sassenage (FR)

Correspondence Address: BIRCH STEWART KOLASCH & BIRCH, PO BOX 747, FALLS CHURCH, VA, 22040-0747, US

(73) Assignees: Universite Joseph Fourier-Grenoble 1, St Martin D'heres (FR); Centre National De La Recherche Scientifique (CNRS), Paris (FR)

(21) Appl. No.: 12/671,432

(22) PCT Filed: Jul. 16, 2008
(86) PCT No.: PCT/EP2008/059276

§ 371 Date: Jan. 29, 2010

(30) Foreign Application Priority Data
Jul. 31, 2007 (FR) 07 56850

(51) Int. Cl. A61H 23/00; A61N 1/18

(52) U.S. Cl. 601/46; 607/134

(57) ABSTRACT

The present invention relates to a tactile stimulation device of an individual, comprising at least three stimulators distributed throughout the buccal cavity, in contact with the gums, the inside of the cheeks and/or the lips so as to communicate three-dimensional information to the individual.

### FIELD OF THE INVENTION

The invention relates to an endo-buccal device for tactile stimulation, able to also include actuation elements.

### BACKGROUND OF THE INVENTION

Tactile stimulation devices have undergone substantial development for several years now. Their purpose is to send information to an individual by means of tactile stimulations, especially electro-tactile or vibro-tactile stimulations.

To this effect, the aim is to implant the device in a region of the body supplied with numerous nerve endings, so as to benefit from optimal receptivity vis-à-vis these stimulations.

With respect to electro-tactile stimulation, devices stimulating the tongue and palate have been developed. In particular, Bach-y-Rita (U.S. Pat. No. 6,430,450) developed a lingual electro-stimulation device comprising a matrix of stimulators implanted in an artificial palate, such that when the tongue makes contact with the palate, information can be transmitted to it. This device benefits from the considerable sensibility of the fore part of the tongue, due to a large number of nerve endings, and on the considerable mobility of this organ which frequently makes spontaneous contact with the palate. This type of device has a wide range of applications, such as compensation for visual deficiencies, taking charge of equilibrium problems (WO 2005/051329), or again prevention of bed sores and plantar ulcers (WO 2006/008406).

However, it presents disadvantages; in particular, the artificial palate takes up considerable space in the buccal cavity and creates difficulties for elocution and swallowing. Its efficacy is further dependent on mobility of the tongue, which varies according to the individuals; now, in the event where the tongue is not very mobile, there is the risk of a considerable time interval between emission of the signal and its perception by the individual.

Also, the matrix of stimulators, such as the palate, is substantially flat, such that it easily transmits spatial bidimensional information (2D), for example of the left/right/front/rear type. In contrast, for transmission of three-dimensional information (3D) also integrating the notions of high/low, it is necessary to resort to 3D information transcoding into a 2D signal. This also involves the individual learning how to make it capable of recognizing this information. It is known that the duration and success of such learning are highly variable from one individual to another.

An electro-stimulation device of the palate has also been developed for blind navigation, also comprising a tactile interface actionable by the tongue (article by Tang and Beebe, An Oral Tactile Interface for Blind Navigation, IEEE Transactions on neural systems and rehabilitation engineering, Vol. 14, No. 1, March 2006, pp. 116-123), but this device also needs an artificial palate with a 2D matrix and therefore has the same drawbacks.

Other stimulation sites have been envisaged, for example, the lower lip (in this respect reference is made to the article by Shim et al, System Development for multichannel electrotactile stimulation on the lips, Medical Engineering & Physics, Vol. 28, No. 7, September 2006, pp. 734-739), but the surface available for stimulation is much smaller, and installation is poorly aesthetical.

Subcutaneous implantation has also been used (especially the subject matter of patent application WO 2005/040989). But this needs surgery for installation and optional removal of the device, and is therefore a much more restrictive method. In addition, implantation is generally done in a substantially flat region of the body, such as the back, the abdomen or the thigh, and is therefore not adapted to transmission of 3D information.

A first aim of the invention is therefore to provide a tactile stimulation device, implanted in a region of the body of major sensibility, but which is not bulky and is aesthetic, and to which actuation elements can be attached. Another aim of the invention is to allow perception of a signal by the individual as soon as it is sent, independently of parameters such as the mobility of the tongue. Another aim of the invention is to develop a tactile stimulation device for transmission of 3D information without transcoding.

### BRIEF DESCRIPTION OF THE INVENTION

For this, the invention proposes a tactile stimulation device of an individual, comprising at least three stimulators distributed throughout the buccal cavity in contact with the gums, the inside of the lips and/or the cheeks of the individual so as to communicate three-dimensional information to said individual.

The device particularly advantageously comprises at least four stimulators not belonging to the same plane.

Stimulation is caused by means of a signal of electric or mechanical pulses.

Each stimulator optionally comprises a matrix of stimulation elements.

The device further comprises a power source, signal-generation means as a function of information to transmit to the individual, and each stimulator comprises receiving means of said signal.

Upstream of the signal-generation means, the device can comprise information-processing means for information originating from sensors.

The different components of the device are particularly advantageously connected by wireless transmission means.

According to a particular embodiment, the power source and/or the receiving means have such dimensions for them to be inserted into an inter-dental space, or into a dental prosthesis.

The device further optionally comprises control means, controlled by the individual, of at least one actuator.

According to a preferred embodiment, the device comprises at least one support capable of being fixed removably on a tooth, and a stimulator, if required the control means of at least one actuator, the receiving means, and/or the power source of said stimulator and, if required, said control means, are solid with said support.

According to another embodiment, the device comprises:

at least one measuring sensor of a physical magnitude or receiving means of external information,

processing means of the measured value or of the information received,

signal-generation means to a stimulator selected from a series of stimulators, as a function of the value or of the information processed,

said external information able to be a coded signal sent by another individual also equipped with a stimulation and actuation device in keeping with the invention comprising at least one control means, the emission of the signal being completed by control means of this device.

The invention also relates to an information communication process by means of a device according to the invention such as defined hereinabove and hereinafter.

### BRIEF DESCRIPTION OF THE DIAGRAMS

Other characteristics and advantages of the invention will emerge from the following detailed description, in reference to the attached figures in which:

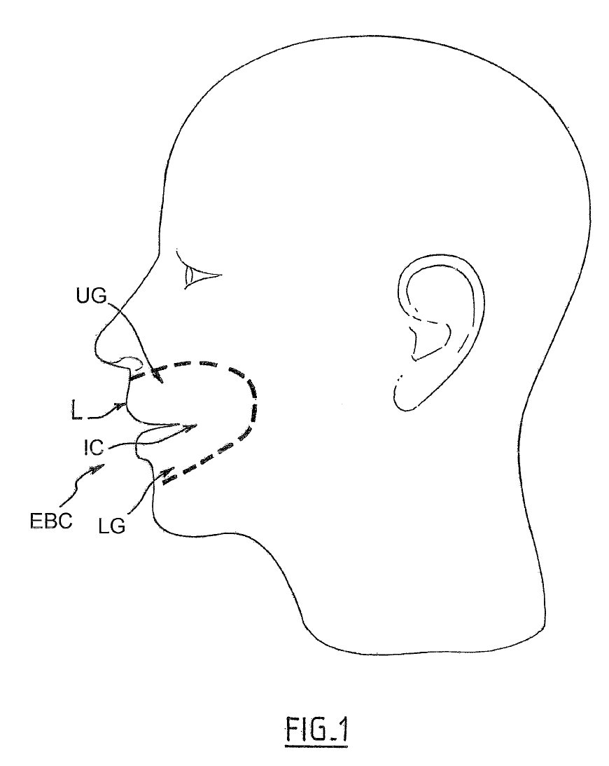

FIG. 1 schematically illustrates the regions of the buccal cavity where the invention can be implemented,

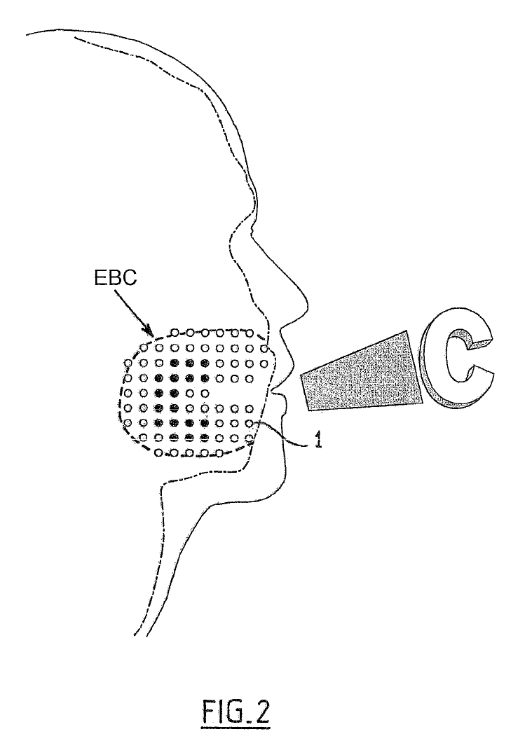

FIG. 2 illustrates an example for implementing the invention within the scope of image restitution (visual assistance),

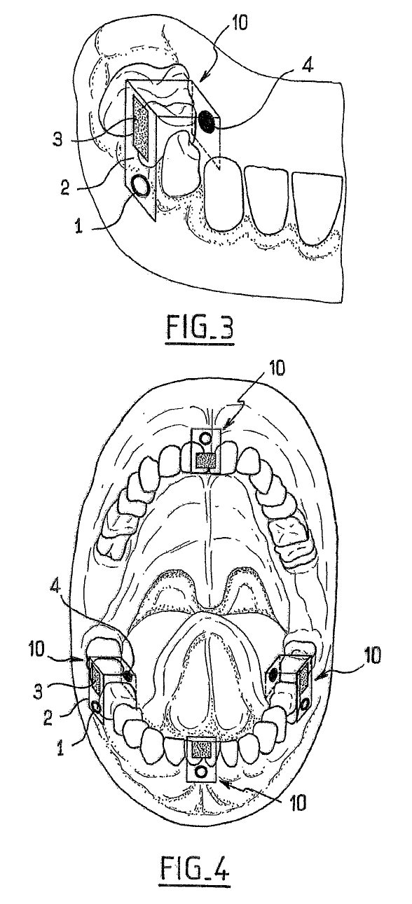

FIGS. 3 and 4 illustrate an onboard device which can be clipped onto one or more teeth.

### DETAILED DESCRIPTION OF THE INVENTION

The device according to the invention is based on electro-tactile or vibro-tactile stimulation of the buccal cavity, and more precisely of the gums or the inside of the cheeks, or even the lips. On FIG. 1, the dotted line schematically delimits the endo-buccal cavity EBC and the regions affected by stimulation: the upper gums UG and lower gums LG, the inside of the cheeks IC and the lips L.

In fact, it works out that these regions of the buccal cavity are almost as sensitive as the palate zone or the tongue, and therefore are well suited to tactile stimulation.

In general, the device according to the invention comprises:
signal-generation means as a function of one or more items of information to transmit to the individual,
at least one stimulator capable of receiving this signal, located inside the buccal cavity, in contact with a gum, the inside of the lips or wall of a cheek,
and at least one power source capable of feeding the signal-generation means, the receiving means (typically, by radio) of the signal and the stimulator.

At least one second stimulator is preferably placed symmetrically in the buccal cavity, allowing transmission to the individual of bidimensional information.

Even more advantageously, there are at least three stimulators in contact with the gums or inside the cheeks or lips (for example, two arranged symmetrically in a horizontal plane and a third arranged above or below this plane) so as to transmit three-dimensional information to the individual.

According to a preferred embodiment, there are at least four stimulators not belonging to the same plane.

In fact, the buccal cavity can be assimilated to a geode, on the surface of which an image is projected. Distributing an appropriate number of stimulators on this curved surface can transmit to the individual information of three-dimensional nature, without transcoding. This is a considerable advantage relative to known devices which are all substantially bidimensional, such as for example matrices of electrodes implanted on the palate and utilised for lingual electro-stimulation, as described in U.S. Pat. No. 6,430,450.

The number of stimulators and their placement in the buccal cavity are defined as a function of the number and nature of information to transmit or, in the particular case where the information is an image, of the level of definition of the image to be restored. The number of stimulators is nevertheless limited by the discriminatory power of the regions on which they are arranged (gums, lips or cheeks), which is of the order of 2 to 3 mm.

Therefore, FIG. 2 represents a head on which the following are delimited by a dashed line: in the left part, the interior of the face, in the right part, the exterior of the face. The endo-buccal cavity EBC is delimited schematically by the dotted line, inside which is a plurality of stimulators 1, electrodes for example. The stimulators 1 are represented by lozenges, the white lozenges corresponding to inactive stimulators, the black lozenges corresponding to the active stimulators for transmitting the 3D image of the letter “C” to the individual.

The stimulation delivered by the device can be electro-tactile or vibro-tactile in nature.

Each stimulator can be localised or be in the form of a matrix comprising a plurality of stimulation elements, which enlarges the stimulation surface. Also, each stimulation element can be fed by a different electric signal (in intensity and/or in frequency), which boosts the amount of information which can be transmitted, by previously defining cartography information on the matrix. As an alternative, all stimulation elements can be fed by the same electric signal.

In the case of electro-tactile stimulation, the stimulator comprises a generator of electric pulses which can be in the form of an electrode or a matrix of electrodes. Typically, a matrix of electrodes comprises around 16 electrodes per cm².

In the case of vibro-tactile stimulation, the stimulator comprises an electromechanical device embedded in a support and able to make it vibrate. Such an electromechanical device is for example a vibrator or piezo-electric actuator, or a matrix of vibrators comprising typically around 16 vibrators per cm².

The person skilled in the art will know how to define the appropriate electrodes or vibrators, especially among those described in U.S. Pat. No. 6,430,450.

The device also and particularly advantageously comprises control means of one or more actuators. The individual can therefore not only receive information, but also execute commands. In this respect, the control means are for example in the form of one or more actuation elements (such as push buttons or contactors) which can for example be pressed using light pressure of the tongue tip. An interface in the form of a keypad with a number of buttons (called “Tongue Touch Keypad”), integrated into an artificial palate, is described for example in the article by Tang and Beebe cited earlier. The pressure of the tongue on the button generates an electric signal which is transmitted to the actuator of the device to be controlled. Concrete examples will be described later.

In keeping with the invention, each of the push buttons is placed in the buccal cavity, for example against a tooth or under the tongue, so as to be readily accessible by the tongue of the individual, as illustrated in FIG. 4, to be described later.

It is also possible to miniaturise the power source and/or the receiving means so as to insert them into an inter-dental cavity, for example inside a dental prosthesis installed in place of a wisdom tooth. Even more advantageously, completely autonomous independent stimulators can be produced: to this effect, a hook to be clipped onto a tooth is integrated, a microprocessor consuming very little energy, a miniature battery, and a stimulator, or even an actuation element.

The stimulators can be placed in the buccal cavity by different means, according to how the device will be used—routine or occasional.

So, for an individual who must be able to be stimulated permanently, as is the case in the replacement of perceptive deficiencies, the stimulators could be adhered to the gums or the inner walls of the lips or cheeks. The choice of appropriate adhesive is within the reach of the person skilled in the art. It is also feasible to fix stimulator supports via suture points.

However, for occasional use—typically, guide or multimedia applications described hereinbelow—a simpler fixing and removal process is preferred. To this effect, one can for example dispose the stimulator on a hook to be clipped onto a tooth or in an inter-dental gap, such that the stimulator is in contact with the gum or the inner wall of the cheek. In this way, the user himself can place and remove the stimulators. Such an onboard device is illustrated in FIG. 3. The onboard stimulation and actuation device 10 comprises, on a support 2 to be clipped onto one or more teeth, a stimulator 1 (for example, an electrode), a microprocessor 3 with a battery, and an actuation element 4 (for example, a push button). The stimulator 1 is placed on the support 2 preferably to the outer side of the gum, so as to be in contact with either the gum, or the inside of the cheek. The actuation element 4 is preferably located to the inner side of the gum, so as to be accessible by the tongue.

According to a preferred embodiment, the signals are transmitted by means of a wireless link, simplifying placing of the device and allowing discrete use. The definition of the transmission means is within the reach of the person skilled in the art.

Operation of the Device

When information is to be transmitted to the individual via stimulators, signals are sent to the latter.

It is specified that the transmitted signals correspond either to raw information, or to processed information.

The information comes from one or more sensors which can form part of the environment of the individual, be attached to him or be attached to another individual. The sensors themselves are known to the expert who will know how to select them as a function of the application. Examples of sensors are cameras, pressure sensors, temperature sensors, cardiac frequency sensors or any other means of analysis of a determined environment, such as for example the physiological component of an individual. The sensors can also measure information not directly perceptible by the individual, such as for example night vision, in military applications, or even the presence of toxic substances. More detailed examples will be described hereinbelow.

“Raw information” is understood as a measurement originating directly from a sensor, which is simply transformed into a pulse signal by the signal generator.

“Processed information” is understood for example as an alert which is triggered only when a predetermined threshold is exceeded. Processing in this case consists of comparison of the measured value by the sensor with this threshold. This stimulates the individual only when a particular situation—especially a risk—must be advised. For example, in the prevention of bed sores, the patient is alerted only when the pressure at the level of the support zone of a member is greater than a given threshold. Processing can also be more complex and can produce information known as “fused”, that is, information resulting from the combination of signals coming from different sensors (for example: pressure, humidity, temperature), and generation of an alert whenever a predetermined threshold is exceeded. This threshold can naturally be adapted as a function of the individual or conditions of use of the device.

As a function of the type of information to be transmitted to the individual, the stimulation device according to the invention could therefore comprise, upstream of the signal generator, a processing unit for information originating from the sensors.

Each signal has coding for activating each of the stimulators. In the case where the stimulators are in the form of matrices of stimulation elements, the signal can be coded so as to activate predefined zones of the matrix. The coding mode is selected as a function of the nature of the information, of the individual and of his capacity to analyse the situation.

According to the case, each stimulation element then emits an electric or vibratory pulse which is perceived by the individual and associated with given information.

The device according to the invention can advantageously be employed in the following applications.

### Example 1

### Substitution or Visual Assistance

A first example of application of the invention concerns visual substitution (for blind people) or visual assistance (for those with poor eyesight: “low vision”). Visual assistance can relate to any person momentarily in a situation of visual deficiency, for example in darkness or fog. The invention can thus apply advantageously to aircraft pilots circling through fog: endo-buccal stimulation can point out their correct position, or even warn them of possible danger (obstacles) or deviation relative to their course.

In this way, the invention proposes using a device (for example coupling of at least two cameras) to reconstitute a 3D image, sampling by a processing unit which breaks down the 3D image into many elements corresponding to “voxels” (in turn projected into a suite of 2D “pixels”, for example projected onto a 3D surface of geode type), each pixel being associated with a stimulator placed in the endo-buccal cavity. The stimulators are particularly advantageously distributed evenly inside the buccal cavity which substantially has the form of a geode. Decomposition of the image into many signals sent to each of the stimulators can then be assimilated into projection of the 3D image onto the buccal geode, as illustrated in FIG. 2.

This projection offers a double advantage.

First, it allows the user to easily interpret the information transmitted, since it retransmits the 3D reality relatively faithfully, without transcoding.

Second, it is known that the regions of the buccal cavity have a discriminating power of the order of 2 or 3 mm, meaning that, for a given surface the number of stimulators which can be implanted is limited. So, in matrices of palate electrodes of the prior art, the matrices have a maximal surface of 4 cm×4 cm, on which a maximum of 12×12 electrodes can be implanted. However, the invention installs stimulators over the entire inner surface of the buccal cavity, which is clearer larger. It is therefore possible to consequently increase the number of stimulators, thus transmitting a finer representation of the 3D image to the individual.

### Example 2

### Replacement of Perceptive Deficiencies

Another example in handicap services concerns the replacement of perceptive deficiencies, in para- or tetraplegic individuals, diabetics, or even amputees. The invention transmits information coming from sensors to the individual.

For example, for immobilised patients likely to suffer from bed sores, pressure sensors located on members in contact with the bed or chair take measurements, which become the subject of processing such that a signal is sent to the patient if measured pressures exceed a predetermined threshold. With the device according to the invention, there are at least four stimulators corresponding to information of right/left/front/rear type, which indicate to the individual how to move the member to prevent bed sores from occurring. Of even greater advantage is that there can be at least one other stimulator giving the individual the command to get up.

This type of application applies particularly well to incorporating at least one actuator control means in the stimulation device, such as described earlier.

A wheelchair can accordingly be fitted with actuators receiving signals transmitted by the control means. Fitting the individual with 6 push buttons can enable him for example to control shifting his chair to the left or the right, to the front or the rear, but also control the rise or fall of the seat or whether it rocks forwards or backwards. According to the preferred number of degrees of liberty, the control means can be placed in the buccal cavity such that they can be actuated by the tongue of the individual as intuitively as possible. This case in point is particularly adapted to the tetraplegic patient whose tongue remains one of the rare organs still mobile.

Another example of the replacement of perceptive deficiencies concerns the fire brigade tending to a fire. Their vest can be fitted with temperature sensors, CO₂ or toxic gas sensors, and information coming from these sensors is sent to them in the form of endo-buccal stimulations to warn them of risk situations.

### Example 3

### Placement, Guiding, or Assistance in 3D Navigation

This example includes a wide diversity of situations.

A first case is that of a diver in deep water, who cannot utilise his vision for orientation. If, as shown in FIG. 4, he is equipped with 4 stimulators located respectively in the left and right, upper and lower regions of the buccal cavity, in this way he can receive indications on the direction in which he should move.

Another case is that of guiding surgical gestures. During a procedure, a surgeon often makes a percutaneous insertion of an instrument, the position of the end of which in the body of the patient he must know precisely. Stimulators placed appropriately in his mouth allow him to communicate information on the inclination he must give to his instrument (to the front/to the left, etc.). The number of stimulators is selected as a function of the amount of information to be transmitted. Similarly, the advantage of endo-buccal stimulation is to allow transmission of additional information to the practitioner operating in an environment already saturated in information: the operation actually stresses his auditory capacity (communication with his assistants), manual ability (handling instruments) and visual ability (operating field and timely surveillance of monitoring screens of the patient).

A third case concerns astronauts, the vestibular organs of which no longer work in weightlessness. Now, it is important for them to know their orientation in space. Currently, they are for example equipped with vests with vibrators which are heavy and give an approximate rendition of reality. Using the device according to the invention, with at least two stimulators, for example with one situated in the upper part and the other in the lower part of the buccal cavity, would point out more conveniently the position of their head (up or down).

Another possible application is guiding a driver via a GPS-type system (“Global Positioning System”), where directions to be taken (relative to the cardinal points or even relative to the road where the car is travelling) are indicated by endo-buccal stimulation. The driver is fitted with an adequate number of stimulators allowing him to transmit all possible directions.

### Example 4

### Multimedia and Leisure Applications

The device according to the invention can advantageously be used in the field of multimedia leisure, such as video games. A game interface communicates three-dimensional information to the player, attracting his attention to an event which can originate from anywhere—for example, by alerting him to the arrival of an object from beyond his field of vision and the provenance of this object. As this type of application supposes that the player is active, the stimulation device will advantageously comprise actuator control means such as joystick.

Still in the multimedia domain, the control means can move a mouse in an office application.

### Example 5

### Communication

Finally, the endo-buccal stimulation and actuation device can be used for communication between two individuals by means of coded language, such as Morse code for example. In this way, the two individuals are fitted with stimulators and actuation elements such as described earlier. The “sender” individual activates certain buttons with his tongue according to learnt coding; the “receiver” individual receives stimulations which he interprets as a low-level message—for example, instructions on moving to the right—or high-level message (type Morse), and can, if required, respond to the other individual by the same means.

Also, the transmitted information cannot be solely spatial information: it is actually possible to communicate other information to the individual: external information—such as a signal emitted by another individual as in the preceding example, or a physical magnitude measured in the environment of the individual. By way of illustration, reference can be made to the example of the surgeon during a procedure. In addition to the spatial orientation of the instrument, important information on the patient can be transmitted, such as for example his temperature, arterial pressure or heart rate. The patient is thus equipped with adequate sensors which measure the selected variables and transmit them to a processing unit. The processing unit takes measurements at a predetermined range of values, associated with one or more stimulators located in the mouth of the surgeon. For example, if the aim is to transmit the information on the temperature of the patient by means of a series of three stimulators, three temperature ranges are defined: low, average, and high. The three stimulators are placed for example one above the other in contact with the inside of the cheek of the surgeon, and, in terms of the one which stimulates him, the surgeon knows if the temperature of the patient is normal or if it increases worryingly.

The device according to the invention therefore has a much smaller footprint than known devices and is arranged in regions of the buccal cavity where it is less likely to disturb buccal functions such as speech or swallowing.

Also, it transmits to the individual information which is denser and more complex than existing devices allow, without resorting to transcoding.

Finally, as is evident, the invention can be utilised is various ways, and the examples described hereinabove are in no way limiting.

1. A tactile stimulation device of an individual, comprising at least three stimulators distributed throughout the buccal cavity, in contact with the gums, the inside of the cheeks and/or the lips so as to communicate three-dimensional information to the individual.

2. The device of claim 1, comprising at least four stimulators not belonging to the same plane.

3. The device of claim 1, wherein the stimulation is caused by means of electric or mechanical signal pulses.

4. The device of claim 1, wherein each stimulator comprises a matrix of stimulation elements.

5. The device of claim 1, further comprising a power source, signal-generation means as a function of information to transmit to the individual, wherein each stimulator comprises receiving means of said signal.

6. The device of claim 5, comprising information-processing means originating from the sensors upstream of the signal-generation means.

7. The device of claim 5, wherein its different components are connected by wireless transmission means.

8. The device of claim 5, wherein the power source and/or the receiving means have such dimensions that they can be inserted into an inter-dental space.

9. The device of claim 8, wherein the power source and/or the receiving means have such dimensions that they can be inserted into a dental prosthesis.

10. The device of claim 1, further comprising control means, by the individual, of at least one actuator.

11. The device of one of claims 5 to 10, comprising at least one support capable of being fixed removably on a tooth, wherein a stimulator, if required control means of at least one actuator, the receiving means, and/or the power source of said stimulator and, if required, said control means, are solid with said support.

12. The device of claim 1, comprising:
at least one measuring sensor of physical magnitude or receiving means of external information,
processing means of the measured value or of the information received,
signal-generation means to a stimulator selected from a series of stimulators, as a function of the value or of the information processed.

13. The device of claim 12, wherein said external information is a coded signal emitted by another individual equipped with a device as claimed in any one of claim 11 or 12 comprising at least one control means, the emission of the signal being done by control means of the device.
